(19) United States
(12) Patent Application Publication
(10) Pub. No.: US 2007/0259011 A1
(43) Pub. Date: Nov. 8, 2007

(54) Oral care products

(75) Inventors: Paul Albert Sagel, Mason, OH (US); Hooman Shahidi, Mason, OH (US); Samuel James St. John, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/455,469

(22) Filed: Jun. 19, 2006

Related U.S. Application Data
(60) Provisional application No. 60/750,455, filed on Dec. 15, 2005.
(60) Provisional application No. 60/701,778, filed on Jul. 22, 2005.

(51) Int. Cl. A61K 8/22; A61K 8/02; A61K 8/24; A61Q 11/00

(52) U.S. Cl. 424/401; 424/49; 424/53; 424/57

(57) ABSTRACT

An oral care product comprising a strip and one or more oral care compositions. The oral care composition comprises an oral care active and when applied to the teeth the oral care product delivers one or more oral care benefits.

### CROSS REFERENCE TO RELATED APPLICATION

This application claims the benefit of U.S. Provisional Application No. 60/750,455, filed Dec. 15, 2005 and U.S. Provisional Application 60/701,778, filed Jul. 22, 2005 both of which are incorporated by reference herein.

### FIELD OF THE INVENTION

The present invention relates to a system and method of delivering oral care actives to oral surfaces.

### BACKGROUND OF THE INVENTION

While tooth whitening products are known in the art, these products are traditionally used by those seeking the cosmetic benefit of whiter teeth. However, there is a different population of consumers who desire whiter teeth and/or more oral benefits including, cleaner teeth, healthier gums, and decreased oral malodor. Therefore, there is a desire to provide oral care products that can deliver oral care benefits in addition to tooth whitening. Additionally, there is a desire to deliver these oral care benefits as part of a daily oral care regimen.

### SUMMARY OF THE INVENTION

A method for delivering one or more oral care benefits to the surfaces of the oral cavity comprising applying a product comprising a substrate and an oral care composition comprising an oral care active to a plurality of teeth and adjacent soft tissue at least once every day for at least about five minutes per application, wherein the product is applied to the teeth daily and adjacent soft tissue for greater than about six months and wherein a fresh product is applied with each use.

A method of reducing tartar on the hard surfaces of the oral cavity comprising applying a product comprising a substrate and one or more oral care compositions to a plurality of teeth and adjacent soft tissue, wherein at least one of the oral care compositions comprises an anti-tartar active at least once a day for at least about five minutes per application, wherein the product is applied to the teeth and adjacent soft tissue daily for greater than about one month, and wherein a fresh product is applied with each use.

### BRIEF DESCRIPTION OF THE DRAWINGS

While the specification concludes with claims which particularly point out and distinctly claim the present invention, it is believed that the present invention will be better understood from the following description of preferred embodiments, taken in conjunction with the accompanying drawings, in which like reference numerals identify identical elements and wherein:

FIG. 1 is a perspective view of an embodiment of an oral care product of the present invention;

FIG. 2 is a cross-sectional view of the oral care product of FIG. 1, taken along line 2-2 thereof;

FIG. 3 is a perspective view of another embodiment of an oral care product of the present invention;

FIG. 4 is a cross-sectional view of the oral care product of FIG. 3, taken along line 3-3 thereof;

FIG. 5 is a perspective view of an embodiment of an oral care product of the present invention;

FIG. 6 is a cross-sectional view of an alternate embodiment of the oral care product of FIG. 3, wherein a plurality of dimples or pockets are illustrated;

FIG. 7 is a cross-sectional view of another alternate embodiment of the oral care product of FIG. 3, wherein the oral care composition is disposed on top of the tooth whitening composition;

FIG. 8 is a cross-sectional view of yet another embodiment of the oral care product of FIG. 3, wherein a barrier composition is shown between the tooth whitening composition and the oral care composition;

FIG. 9 is a cross-sectional view of a dental arch illustrating the product of FIG. 3 applied to the facial and lingual surfaces of a plurality of teeth;

FIG. 10 is a cross-sectional view of the dental arch of FIG. 9 taken along line 8-8 thereof; illustrating the oral care product of FIG. 3 applied to some soft tissue adjacent the facial and lingual surfaces of a plurality of teeth;

FIG. 11 is a perspective view of another embodiment of the product of FIG. 3, illustrating an optional release liner;

FIG. 12 is a perspective view of another embodiment of the product of FIG. 3, illustrating an optional release liner;

FIG. 13 is a perspective view of another embodiment of the product of FIG. 3, illustrating an optional release liner; and

FIG. 14 is a perspective view of another oral care product of the present invention, wherein various patterns of inter-mixing the tooth whitening composition and the oral care composition are shown.

### DETAILED DESCRIPTION OF THE INVENTION

The abbreviation “cm”, as used herein, means centimeter. The abbreviation “mm” as used herein, means millimeter. The abbreviation “mg”, as used herein, means milligram. The abbreviation “g”, as used herein, means gram.

As used herein the term oral care composition means any substance that contains one or more active(s) at a level wherein upon use, delivers an oral benefit sought by the wearer without detriment to the oral surface it is applied to. The oral care composition can address one or more oral conditions with one or more oral care actives. Examples of oral conditions these oral care actives address include, but are not limited to, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores and tooth abscesses, oral malodor, dental erosion, gingivitis, and/or periodontal disease. Oral conditions are further described in WO 02/02096A2 published Jan. 10, 2002, P&G.

As used herein, the phrase “oral care active” or “active” is intended to refer to any material that is safe for use in the oral cavity that provides changes to the overall health of the oral cavity, and specifically the condition of the oral surfaces the oral care substance contacts including, but not limited to, tooth whitening, anti-tartar, anti-caries, and/or anti-microbial actives or any combination thereof.

By “safe and effective amount”, as used herein, is meant an amount of an active at high enough levels to significantly improve the condition to be treated, but low enough to avoid serious side effects (at a reasonable benefit/risk ratio), within the scope of sound medical/dental judgment. The safe and effective amount of an active may vary with the particular condition being treated, the age and physical condition of the patient being treated, the severity of the condition, the duration of treatment, the nature of concurrent therapy, the specific form of the source employed, and the particular vehicle from which the active is applied.

All percentages and ratios used hereinafter are by weight of total oral care composition, unless otherwise indicated.

All measurements referred to herein are made at 25° C. unless otherwise specified.

All percentages, ratios, and levels of ingredients referred to herein are based on the actual amount of the ingredient, and do not include solvents, fillers, or other materials with which the ingredient may be combined as a commercially available product, unless otherwise indicated.

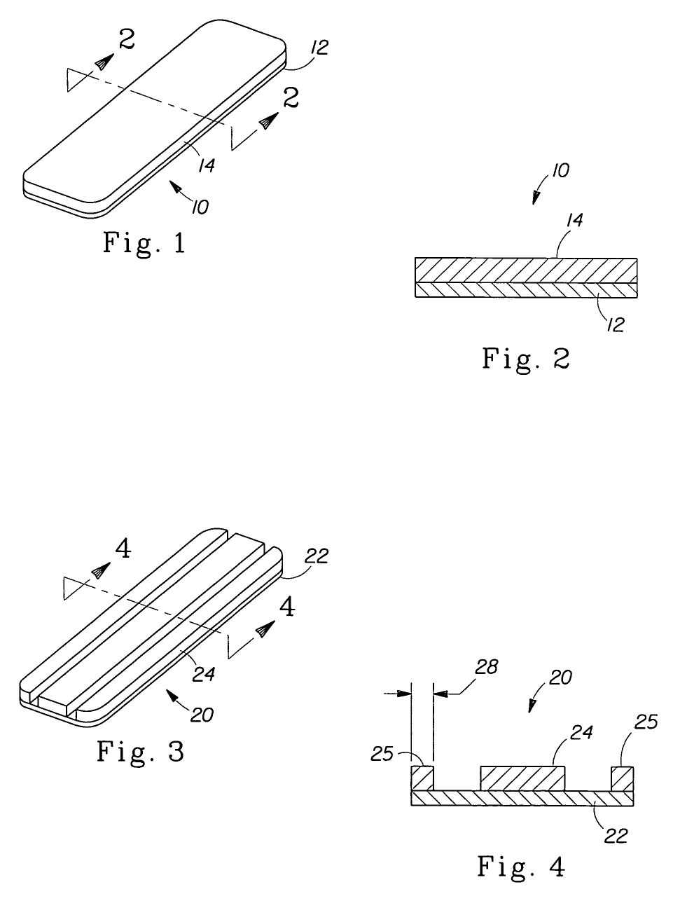

Referring now to the drawings, and more particularly to FIGS. 1 and 2, there is shown a first embodiment of the present invention, generally indicated as 10, representing a product for providing an oral care composition to a plurality of teeth. This oral care composition can be a single composition which provides one or more oral benefits. The strip of material 12 can be coated with an oral care composition 14. This oral care composition comprises an active. In one embodiment the oral care active is peroxide and the oral benefit(s) provided include tooth whitening and anti-tartar. The oral care product 10 may comprise a substrate, such as a strip of material 12, which may be initially substantially flat, and may have rounded corners. While the strip of material 12 may be initially substantially flat, it is contemplated that the strip of material may be provided in other forms. The strip of material 12 can have shapes and sizes, such as described for example in U.S. Pat. No. 6,884,426.

In another embodiment, shown as 20 (FIGS. 3-5), the product provides multiple oral care benefits, one of which is tooth whitening, with multiple oral care compositions which comprise one or more oral care actives or one oral care composition comprising multiple oral care actives. In this embodiment the product provides an oral care composition having a tooth whitening active to a plurality of teeth and an oral care composition having an oral care active that provides a non-whitening oral care benefit including, but not limited to anti-tartar, anti-caries, antimicrobial etc. . . . that is applied to soft tissue adjacent the plurality of teeth. The oral care product 20 may comprise a substrate, such as a strip of material 22, which may be initially substantially flat, preferably with rounded corners. While the strip of material 22 may be initially substantially flat, it is contemplated that the strip of material may be provided in other forms.

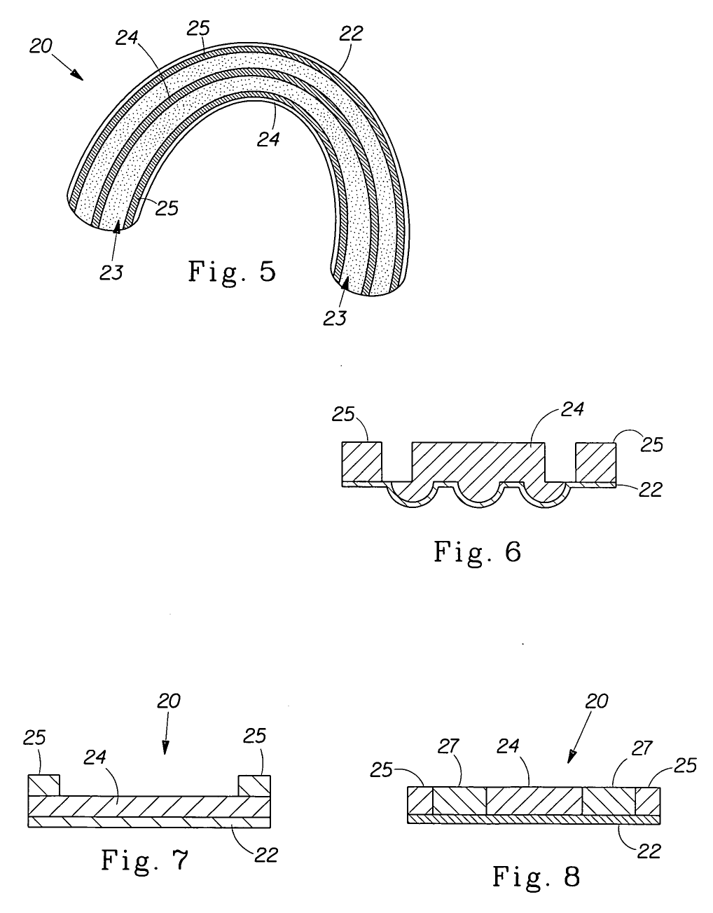

In all the embodiments of the present invention described herein, the strip of material can be provided in an arched shape or the shape of a tray having a trough 23, such as described in USPNs 2005/0089821, 2004/0241616, and 2005/0089819 and as shown by way of example in FIG. 5. The strip of material 22 can also have other shapes and sizes, such as described for example in U.S. Pat. No. 6,884,426. The strip of material 22 can include shallow pockets or dimples as described in U.S. Pat. No. 6,136,297 and as shown by way of example in FIG. 4. Alternatively, it is contemplated that the product can be provided in a form that does not include a substrate and which merely comprises an oral care composition, such as a tooth whitening composition, for example as described in U.S. Pat. No. 6,419,906.

In one embodiment as shown in FIGS. 1 and 2, an oral care composition 14 is applied or coated onto strip of material 12. The oral care composition 14 is provided in the form of a stripe disposed along at least a portion of the strip of material 12. In one embodiment, the oral care composition extends substantially along the longitudinal length of the strip of material. The oral care composition may be provided in a shape and size so that it is applied only to the facial surfaces of a plurality of teeth. Alternatively, the oral care composition may be provided in a shape and size so that it contacts both the facial and lingual surfaces of a plurality of teeth during use and/or the soft tissue. For example, the oral care composition coats the strip which is shaped to cover the facial surface of the plurality of teeth wrap, wrap over the incisal edge of the teeth and cover the lingual surface of the plurality of teeth. In another embodiment, two oral care compositions one of which provides a tooth whitening benefit, are disposed as stripes on the strip of material 22 (as shown in FIGS. 3-5). One of the oral care composition(s) 25 may be applied near, adjacent, or along one or both of the peripheral edges of the strip of material 22 so that the oral care composition contacts at least some of the soft tissue adjacent the plurality of teeth during use. The oral care composition may or may not also contact the hard tissue of the plurality of teeth during use. In this same embodiment the oral care composition that provides a tooth whitening benefit contacts the hard tissue of the plurality of teeth.

In one embodiment, the oral care composition may be provided as a single stripe that is intended to contact at least some of the soft tissue adjacent the facial surfaces of the plurality of teeth. In another embodiment, the oral care composition is provided as two or more stripes wherein at least one stripe is intended to contact at least some of the soft tissue adjacent the facial surfaces of the plurality of teeth, and at least one stripe is intended to contact the lingual surface of the plurality of teeth. The oral care composition may be provided in a form having a width 28 between about 1 mm and about 5 mm. Where two or more stripes of the oral care composition are provided, it is contemplated that distinct stripes may incorporate distinct oral care actives. For example, one stripe might contain a first oral care active while a second stripe might contain a second oral care active distinct from the first oral care active. While multiple oral care compositions are shown as continuous stripes in FIGS. 3-5, it is contemplated that they can be provided as discontinuous stripes, a plurality of stripes, or in other regular or irregular shapes such as dots. Alternatively, one oral care composition may be coated over a substantial portion of the strip of material and second oral care composition may be placed on top of the first oral care composition, as shown by way of example in FIG. 7. In some embodiments, such as shown in FIGS. 3, 4, 5, 6, and 7, the multiple oral care compositions are distinct. As used herein, distinct is intended to refer to compositions which have generally defined boundaries or interfaces between the compositions as opposed to a homogenous mixture. Some intermixing at the boundaries or interface is contemplated and considered within the concept of distinct compositions. As used herein, distinct as it refers to actives is intended to refer to actives that are not identical in formula or composition.

Further, while the oral care composition has been described as placed near the peripheral edge of the strip of material, it is also contemplated that it can be located elsewhere in or on the strip of material. For example, an oral care composition can comprise oral care active intermixed with the polymer material forming the strip of material. One or more oral care composition(s) may be preapplied to the strip of material 22 by the manufacturer and then packaged in a pouch or other suitable container for shipment.

In one embodiment two oral care compositions, each of which comprises an one or more oral care actives are applied to a strip. In this embodiment one of the oral care compositions comprises a tooth whitening active, and the two oral care compositions are distinct. As shown in FIG. 8, it may be desirable to provide a barrier composition 27 between the oral care composition comprising the tooth whitening active 24 and second oral care composition 25. The barrier composition may be used to keep the oral care composition comprising the tooth whitening active 24 and the second oral care composition 25 separate during manufacture, storage, and/or use. The barrier composition can also be provided in the form of a stripe, although other forms are contemplated. The barrier composition can be formed from a gelling or adhesion agents described herein. In one embodiment the barrier composition does not contain any oral care active.

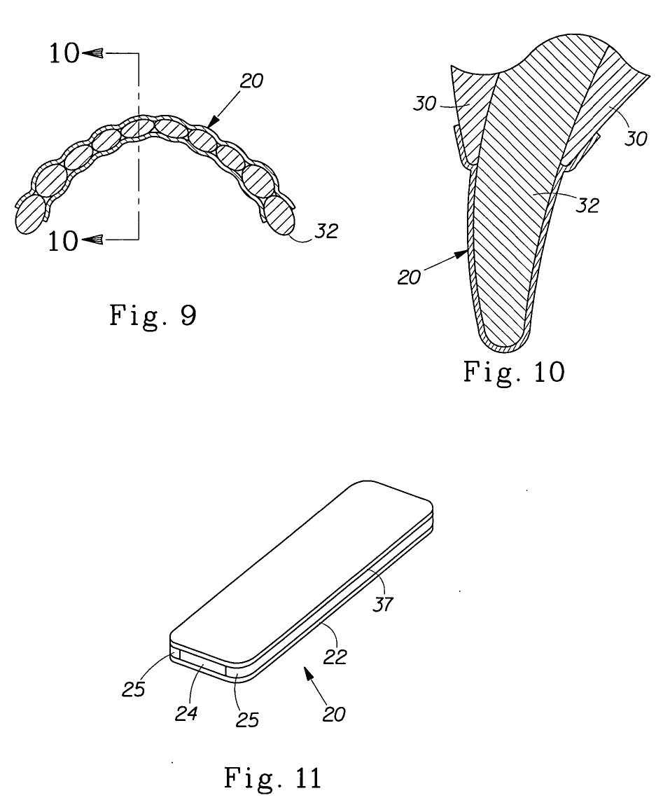

Referring to FIGS. 9 and 10 the product 20 is illustrated as applied to the facial and lingual surfaces of a plurality of adjacent teeth 32 as well as some of the soft tissue 30 adjacent the facial and lingual surfaces of the plurality of teeth. The soft tissue may refer to any soft tissue adjacent or between the teeth, including but not limited to the papilla, marginal gingiva, gingival sulculus, inter dental gingiva, gingival gum structure on lingual and buccal surfaces up to and including muco-gingival junction and the pallet. While the product 20 is shown as applied to both the facial and lingual surfaces and some soft tissue adjacent thereto, it is contemplated that the product 20 may be sized for application to fewer surfaces than shown. For instance, the product 20 might be sized for application to only the facial surfaces and the soft tissue adjacent thereto or, in another embodiment, to the facial surfaces, and the soft tissue adjacent thereto, and a portion of the lingual surfaces but not the soft tissue adjacent the lingual surfaces.

The strip of material 22 may have a thickness and flexural stiffness which enables it to conform to the contoured surfaces of tooth 32 and to the soft tissue. The strip of material may also be readily conformable to the interstitial spaces between the plurality of teeth. In one embodiment, the strip of material of can conform to the tooth surfaces and interstitial spaces without permanent deformation. While the strip of material may have sufficient flexibility to conform to the interstitial spaces between teeth, it is contemplated that the strip of material can have other degrees of flexibility. For example, in one embodiment, the strip of material may only have sufficient flexibility to conform to the general contours of the facial and lingual surfaces of the teeth while not having sufficient flexibility to conform to the interstitial spaces between the teeth or the strip of material might permanently deform (e.g., if provided as a wax type material) when conformed to the contours of a tooth surface. It is also contemplated that the strip of material 12 can have the aforementioned thickness and flexural stiffness.

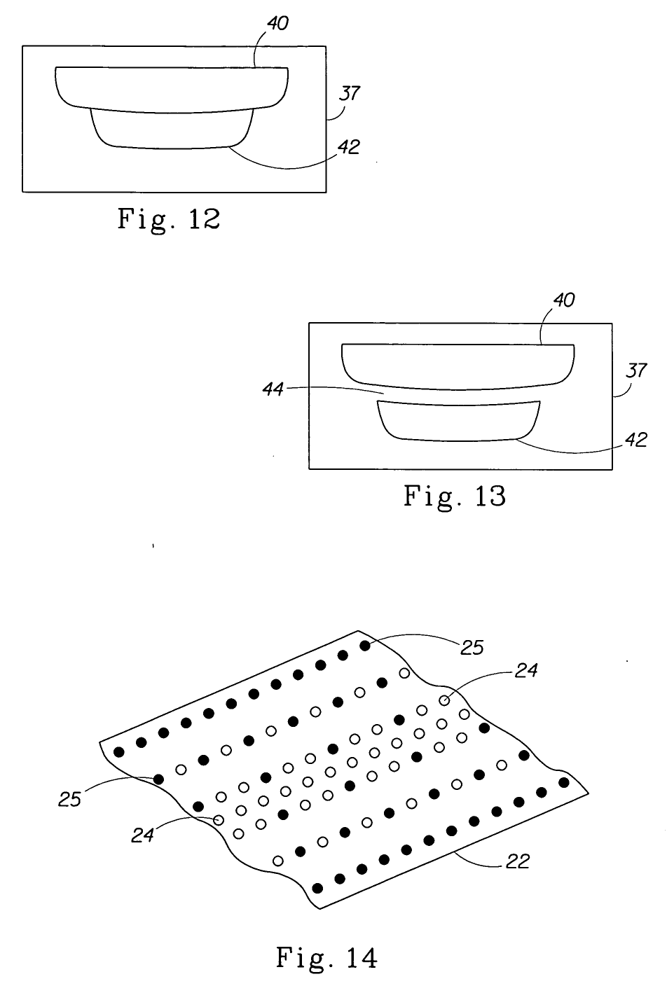

Referring to FIG. 11, an optional release liner 37 can be provided with the product 20. The release liner 37 is disposed adjacent the oral care composition 24 and/or the oral care composition 25. The optional release liner 37 is removed prior to application of the product 20 to the teeth and soft tissue. It is also contemplated that product 10 can have a similar optional release liner. Additionally, (as shown in FIGS. 12-13) in one embodiment two products can be placed on the release liner 37 adjacent to one another, one product shaped for the upper dentition 40 and one product shaped for the lower dentition 42. In another embodiment, two products can be placed on the release liner 37 with a space 44 between the product shaped for the upper dentition 40 and the product shaped for the lower dentition 42.

While the above-described embodiments illustrate the tooth whitening composition and oral care composition as distinct compositions that are not intermixed, FIG. 14 illustrates a perspective view of an embodiment where the tooth whitening composition and the oral care composition are still distinct but are intermixed. As seen in FIG. 14, a tooth whitening composition 24 is provided as a plurality of dots intermixed with an oral care composition 25 provided as a plurality of dots. The dots may be provided as a regular repeating pattern, as an irregular pattern, or combination thereof. The dots may be applied to the strip of material using a printing process that prints the dots onto the strip of material. While dots are shown, it is contemplated that a wide variety of other shapes (both regular and irregular) and sizes may be provided. Some processes that may be suitable for use with the present invention are solution printing, screen printing, gravure printing, stencil printing, inkjet printing, examples of which are described in USPNs 2005/0128260; U.S. Pat. Nos. 6,874,862; and 5,636,566. The oral care composition can be printed as solutions or semi-viscous solutions which are dried to form the dot or other structure.

Strip of Material

The strip of material as described herein serves as a protective barrier for the oral care composition. It can prevent substantial leaching and/or erosion of the oral care composition by for example, the wearer's lips, tongue, as well as saliva. This allows the oral care active to act upon their oral surfaces for an extended period of time, from several minutes to several hours.

The strip of material may comprise any material, including one or more polymers, wax, foam, natural and synthetic woven materials, non-woven material, foil, paper, rubber, and combinations thereof. The strip of material can be provided as a polymeric film. The strip of material may be a single layer of material or a laminate comprising more than one layer. For example, the strip of material may comprise a laminate of two or more polymeric films or may comprise a wax and non-woven material, such as a polymeric scrim or mesh as described in USPN 2004/0005277. The strip of material may be substantially water impermeable. Some polymers suitable for use in manufacturing the strip of material include, but are not limited to, polyethylene, ethylvinylacetate, polyesters, ethylvinyl alcohol and combinations thereof. Examples of polyesters include Mylar® and fluoroplastics such as Teflon®, both manufactured by DuPont. The strip of material may have a thickness less than about 1 mm, or less than about 0.05 mm, or between about 0.001 and about 0.03 mm.

The strip of material may have any shape and size that covers the facial and/or lingual surfaces of a plurality of teeth and/or some of the soft tissue adjacent the facial and/or lingual surfaces of a plurality of teeth. In one embodiment, the length of the strip of material is from about 2 cm to about 12 cm and preferably from about 4 cm to about 9 cm. The width of the strip of material will also depend upon the oral surface area to be covered. In one example, the width of the strip of material is from about 0.5 cm to about 4 cm and preferably from about 1 cm to about 2 cm. As seen in FIG. 8, the strip of material may be folded about or is otherwise disposed about the incisal edges of the plurality of teeth. The overall thickness of the product 10 may be less than about 2 mm, or less than about 1 mm, or less than about 0.5 mm prior to use and/or during use.

The flexural stiffness of the strip of material may be measured by using a strain gauge affixed to the end of a horizontal beam. The opposite end of the beam presses across a strip of the sample to force a portion of the strip into a vertical groove in a horizontal platform upon which the sample rests. A micrometer, wired to the strain gauge is calibrated in grams of deflection force. The rigidity of the sample is read directly from the micrometer and expressed as grams per centimeter of sample strip width. In some embodiments, the strip of material may have a flexural stiffness of less than about 5 grams/cm as measured on a Handle-O-Meter, model #211-300, available from Thwing-Albert Instrument Co. of Philadelphia, Pa., as per test method ASTM D2923-95. In other embodiments, the strip of material has a flexural stiffness less than about 3 grams/cm, or less than about 2 grams/cm, or between about 0.1 grams/cm and about 1 grams/cm.

A low flexural stiffness can allow the strip of material to conform to the contours of the oral surfaces of the wearer's mouth because there is little residual force within the strip of material to cause it to return to its shape just prior to its application to the oral surface. The strip of material's flexibility can enable it to contact soft tissue over an extended period of time without irritation, and the strip of material would not require pressure significant pressure to conform it to the surfaces of the teeth and/or soft tissue.

The strip of material is held in place on the oral surface by adhesive attachment provided by one or more of the oral care compositions, the barrier composition or any other composition or active/agent that is applied to, coated on, or intermixed with the strip of material. Alternatively, the strip of material can be held in place by mechanical pressure from deforming the strip of material about the facial and/or lingual surfaces of the teeth. For example, where the strip of material undergoes permanent deformation during application to the teeth, the strip of material may be held in place by the pressure or fit of the strip of material about the teeth. In some embodiments, the strip of material may be dissolvable or erodible during use. Examples of strips of material that are dissolvable or erodible during use are described in U.S. Pat. No. 6,649,147; and U.S. Patent Appl. Nos. 2003/0228264; and 2004/0062724. The strip of material may dissolve or erode in less than about 1 hour, less than about 30 minutes, less than about 15 minutes or less than 5 minutes. After dissolution or erosion of the strip of material, the oral care composition may be left behind on the facial and/or lingual tooth surfaces and/or the soft tissues surfaces to can continue to act upon those surfaces.

In some embodiments having multiple oral care compositions, it may be desirable for the oral care compositions to then dissolve or erode at differing rates. For example, after dissolution of strip of material (or removal of the strip of material), an oral care composition comprising a tooth whitening active may rapidly dissolve while the oral care composition comprising an antibacterial active dissolves more slowly to allow the oral care active to continue to act upon the soft tissue or to deliver an active to the oral cavity for a longer period of time. Examples of an oral care composition that could quickly dissolve upon removal, dissolution, or erosion of the strip are disclosed U.S. Pat. Nos. 6,669,930 and 5,098,303. It may be desirable for the oral care composition to remain on the oral tissue for up to 2 days. In another embodiment, it may be desirable for the oral care composition to remain on the oral tissue for greater than about 30 minutes, 1, 2, 4, 6, 8, 10, 12, 14, 16, 18, or 20 hours and or less than 24, 20, 18, 16, 14, 12, 10, 8, 6, or 4 hours and any combination thereof. Examples of oral care compositions that could adhere to tissues of the oral cavity for extended periods of time are disclosed in USPNs 2005/0100515 and U.S. Pat. No. 6,649,147.

Where the strip of material is non-dissolving or non-erodible, the strip of material and the one or more oral care compositions may be cleanly removed from the tooth surfaces and soft tissue so that little or no residue of the oral care composition(s) are left behind upon removal. Alternatively, removal of the strip of material may cleanly remove the one oral care composition, such as a tooth whitening oral care composition, and leave a second oral care composition on the oral tissue so that the oral care active in the second oral care composition can continue to act upon the tissue over an extended period of time. In the later instance, the second oral care composition might have less adhesive affinity for the strip of material than the first oral care composition has for the strip of material so that the second oral care composition remains behind while the first oral care composition adheres to the strip of material and is removed therewith. The strip of material might have a release coating (e.g., Teflon, silicone, fluoropolymers, etc.) applied thereto over the portion of the strip of material that contacts the second oral care composition to facilitate or effectuate the release between the strip of material and the second oral care composition upon removal of the strip of material from the teeth.

The peel force required to remove the strip of material from the tooth surfaces and the soft tissue may be between about 1 gram and about 50 grams for a 1.5 cm strip width (approximately 17 grams/cm) is all that is required. In another embodiment, the peel force may be between about 10 grams and about 40 grams, or between about 20 grams and about 30 grams.

Referring to FIG. 11, the strip of material 12 may also be perforated. Perforations can be provided adjacent or below the oral care composition 15 for example where it is desirable for the oral care active to more readily dissolve or otherwise diffuse into the oral cavity. The perforations can be provided in a regular or irregular pattern.

Oral Care Compositions

An oral care composition of the present invention comprising a safe and effective amount of an oral care active may be topically applied to the mucosal tissue of the oral cavity, to the gingival tissue of the oral cavity, to the surface of the teeth and/or any combination thereof for the treatment or prevention of the above mentioned conditions of the oral cavity, in several ways. The amount of oral care substance applied to the strip of material or oral surface depends upon the size and capacity of the piece of material, concentration of the active, and the desired benefit sought. Generally, less than about 1 gram of oral substance is used. In one embodiment from about 0.05 grams to about 0.5 grams of the oral care substance is used. In another embodiment from about 0.1 to about 0.4 grams of the oral care substance is used. The oral care composition can include an oral care active that delivers one or more benefits to the soft tissue, or, in some instances to the hard tissue of the teeth. The oral care active can be any material that is generally recognized as safe for use in the oral cavity that provides changes to the overall health of the oral cavity, and specifically the condition of the oral surfaces the oral care active contacts. The level of oral care active in the oral care composition of the present invention is from about 0.01% to about 40% in one embodiment. In another embodiment the level of oral care active in the oral care composition is from about 0.1% to about 20%. In another embodiment the level of oral care active in the oral care composition is from about 0.5% to about 10%, and in yet another embodiment the level of oral care active in the oral care composition is from about 1% to about 7%, and in yet another embodiment the level of oral care active in the oral care composition is from about 0.5% to about 5%. Benefits can include tooth whitening and/or bleaching, anti-microbial or bacteriocidal, anti-tartar, anti-caries, anti-sensitivity, malodor protection, etc. The oral care composition can comprise one or multiple oral care actives. In one embodiment of the present invention the oral care composition comprises one oral care active that delivers multiple oral care benefits. In another embodiment of the present invention the oral care composition comprises multiple oral care actives each of which delivers a distinct oral care benefit. In another embodiment the oral care composition comprises from about 0.5% to about 5% of a non-whitening oral care active and from about 1% to about 30% of a whitening oral care active. In another embodiment the oral care composition comprises from about 0.5% to about 3% of a non-whitening oral care active and from about 1% to about 10% of a whitening oral care active. In one embodiment the oral care composition comprises from about 1% to about 3% pyrophosphate by weight of the total oral care composition and from about 1% to about 10% of peroxide by weight of the total oral care composition.

It is also contemplated that a single oral care product can comprise multiple oral care compositions, each of which comprises one or more oral care actives. Some oral care actives that are suitable for use in the oral care composition are discussed more fully below.

The oral care composition can include one or more gelling agents, which may also act as an adhesive agent to adhere the oral care composition to the plurality of teeth. The concentration of the gelling agent may be greater than about 2, 4, 6, 8, 10, 15, 20, 30, 40, 50, 60 or less than about 80, 70, 60, 50, 40, 30, or 20 percent by weight of the oral care composition.

Suitable gelling agents, adhesion agents, and/or oral care compositions useful in the present invention are described in U.S. Pat. Nos. 6,649,147; 6,780,401; 2004/0102554; 2005/0089819; 2003/0152528; 6,419,906; and 2005/0100515. Some of the gelling agents or adhesion agents may include silicone, polyethylene oxide, polyvinyl alcohol, poly alkyl vinyl ether-maleic acid copolymer (PVM/MA copolymer) such as, Gantrez AN 119, AN 139, and S-97, polyvinyl alcohol, polyacrylic acid, Poloxamer 407 (Pluronic), polyvinyl pyrrolidone-vinyl acetate copolymer (PVP/VA copolymer), such as Luviskol VA, and Plasdone S PVP/VA, polyvinyl pyrrolidone (PVP, e.g., K-15 to K-120), Polyquaterium-11 (Gafquat 755N), Polyquaterium-39 (Merquat plus 3330), carbomer or carboxypolymethylene (Carbopol), hydroxy propyl methyl cellulose, hydroxy ethyl cellulose, hydroxy propyl cellulose, carboxymethyl cellulose, gelatin and alginate salt such as sodium alginate, natural gums such as gum karaya, xanthan gum, Guar gum, gum arabic, gum tragacanth, and mixtures thereof.

A humectant or plasticizer may be included in the oral care composition, including glycerin, sorbitol, polyethylene glycol, propylene glycol, and other edible polyhydric alcohols. The humectants may be present between about 10% to about 95%, or between about 50% and about 80%, by weight of the oral care composition. An oral care composition can also include flavoring agents, sweetening agents, opacifiers, and coloring agents.

Water may be included in the oral care composition. The concentration of water may be greater than about 0, 2, 5, 10, 15, 20, 25, 30, 35, 40, or 45% and/or less than about 80, 70, 60, 50, or 40% by weight of the oral care composition and any combination thereof.

1. Tooth Whitening Actives

Generally, less than about 1 gram of an oral care composition comprising a tooth whitening active is applied to the strip of material. In one embodiment, between about 0.05 grams and about 0.5 grams, or between about 0.1 gram to about 0.4 grams of the oral care composition comprising a tooth whitening active is used. The amount of oral care composition comprising a tooth whitening active per square cm of material may be less than about 0.2 grams/cm², preferably from about 0.005 to about 0.1 grams/cm², and more preferably from about 0.01 grams/cm² to about 0.04 grams/cm².

The oral care composition comprising a tooth whitening active can be provided in a variety of forms, such as a solid, semi-solid or a liquid, including a viscous liquid, a paste, a gel, or a solution. Preferably, the oral care composition comprising a tooth whitening active is in the form of a gel. The oral care composition comprising a tooth whitening active may have a viscosity between about 200 and about 5,000,000 cps at low shear rates (less than one 1/seconds). In one embodiment, the viscosity may be between about 100,000 and about 1,500,000 cps and in another embodiment between about 400,000 and about 1,000,000 cps.

A polymeric mesh or scrim may be incorporated in the oral care composition comprising a tooth whitening active.

The tooth whitening or bleaching actives that may be suitable for use in the oral care composition include peroxides, metal chlorites, perborates, percarbonates, peroxyacids, and combinations thereof. Suitable peroxide compounds include hydrogen peroxide, calcium peroxide, carbamide peroxide, and mixtures thereof. Suitable metal chlorites include calcium chlorite, barium chlorite, magnesium chlorite, lithium chlorite, sodium chlorite, and potassium chlorite. Additional whitening actives may be hypochlorite and chlorine dioxide. The oral care composition may contain a tooth whitening active at greater than about 2, 4, 6, 8, 10, 15, 20% and/or less than about 40, 25, 20, 18, 16, 14, 12, or 10% by weight of the oral care composition.

It is contemplated that any one of and/or combination of the embodiments of the present invention can be used daily as part of an oral care regimen. Using these oral care compositions of the present invention as part of a daily oral care regimen allows a user to achieve and sustain a variety of desired oral care benefit, including but not limited to white, tartar free teeth. However, if the oral care composition includes peroxide, and the oral cavity is exposed to a composition comprising a peroxide substance every day, it is desirable to use an oral care composition that is highly tolerable to the surfaces of the oral cavity and minimizes the generation of tooth sensitivity. An example of an oral care composition that is tolerable to the soft tissue of the oral cavity and minimizes the generation of tooth sensitivity, includes one with a peroxide concentration of less than about 7.5% peroxide by weight of the total tooth whitening composition and a peroxide density of less than about 1.3 mg/cm². In one embodiment the tooth whitening composition has a peroxide concentration of greater than about 0.01% and less than about 7.5% peroxide by weight of the tooth whitening composition and a peroxide density of less than about 1.3 mg/cm². In another embodiment the tooth whitening composition has a peroxide concentration of greater than about 6.5% and less than about 7.5% peroxide by weight of the tooth whitening composition and a peroxide density of less than about 1.3 mg/cm². In another embodiment the tooth whitening composition has a peroxide concentration of greater than about 6.0% and less than about 6.5% peroxide by weight of the tooth whitening composition and a peroxide density of less than about 1.3 mg/cm², and in yet another embodiment the tooth whitening composition has a peroxide concentration of less than about 6.0% peroxide by weight of the tooth whitening composition and a peroxide density of less than about 1.3 mg/cm²

Peroxide density is the ratio of the amount of peroxide active (mg) or peroxide dose to the surface area (cm²) of the thin layer that is applied to the tooth surfaces and adjacent soft tissue of the oral cavity. Peroxide density and the calculation thereof is discussed in U.S. Pat. No. 6,949,240. In most instances, the surface area of the thin layer can be approximated by the surface of the strip of material if the entire strip of material is applied to the oral cavity and if the entire strip of material is coated with the thin layer of the tooth whitening composition. For example, if the strip of material is rectangular and has a length of 6.5 cm and width of 1.54 cm and the thin layer of tooth whitening composition is coated over an entire side of the strip of material, the total surface area is 10 cm². If the tooth whitening composition contains 6.5% hydrogen peroxide and the strip of material contains 0.2 g of the tooth whitening composition, then the hydrogen peroxide dose is 13 mg. The corresponding peroxide density is 1.3 mg/cm².

2. Anti-Tartar Actives

Anti-tartar actives known for use in dental care products includes phosphates. Phosphates include pyrophosphates, polyphosphates, polyphosphates and mixtures thereof. Pyrophosphates are among the best known for use in dental care products. Pyrophosphate ions are delivered to the teeth derive from pyrophosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. While any of the above mentioned pyrophosphate salts may be used, tetrasodium pyrophosphate salt is preferred. In one embodiment the oral care composition comprises from about 0.5% to about 5% of a pyrophosphate by weight of the oral care composition. In another embodiment the oral care composition comprises from about 0.5% to about 3% of a pyrophosphate by weight of the oral care composition.

The pyrophosphate salts are described in more detail in Kirk & Othmer, Encyclopedia of Chemical Technology, Third Edition, Volume 17, Wiley-Interscience Publishers (1982), incorporated herein by reference in its entirety, including all references incorporated into Kirk & Othmer. Additional anticalculus actives include pyrophosphates or polyphosphates disclosed in U.S. Pat. No. 4,590,066 issued to Parran & Sakkab on May 20, 1986; polyacrylates and other polycarboxylates such as those disclosed in U.S. Pat. No. 3,429,963 issued to Shedlovsky on Feb. 25, 1969 and U.S. Pat. No. 4,304,766 issued to Chang on Dec. 8, 1981; and U.S. Pat. No. 4,661,341 issued to Benedict & Sunberg on Apr. 28, 1987; polyepoxysuccinates such as those disclosed in U.S. Pat. No. 4,846,650 issued to Benedict, Bush & Sunberg on Jul. 11, 1989; ethylenediaminetetraacetic acid as disclosed in British Patent No. 490,384 dated Feb. 15, 1937; nitrilotriacetic acid and related compounds as disclosed in U.S. Pat. No. 3,678,154 issued to Widder & Briner on Jul. 18, 1972; polyphosphonates as disclosed in U.S. Pat. No. 3,737,533 issued to Francis on Jun. 5, 1973, U.S. Pat. No. 3,988,443 issued to Ploger, Schmidt-Dunker & Gloxhuber on Oct. 26, 1976 and U.S. Pat. No. 4,877,603 issued to Degenhardt & Kozikowski on Oct. 31, 1989; all of these patents are incorporated herein by reference. Anticalculus phosphates include potassium and sodium pyrophosphates; sodium tripolyphosphate; diphosphonates, such as ethane-1-hydroxy-1,1-diphosphonate, 1-azacycloheptane-1,1-diphosphonate, and linear alkyl diphosphonates; linear carboxylic acids; and sodium zinc citrate.

Actives that may be used in place of or in combination with the pyrophosphate salt include such known materials as synthetic anionic polymers including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Other anti-tartar actives include sodium hexametaphosphate.

3. Anti-Caries Actives

Fluoride ion sources are well know for use in oral care compositions as anticaries Actives. Fluoride ions are contained in a number of oral care compositions for this purpose, particularly toothpastes. Patents disclosing such toothpastes include U.S. Pat. No. 3,538,230, Nov. 3, 1970 to Pader et al; U.S. Pat. No. 3,689,637, Sep. 5, 1972 to Pader; U.S. Pat. No. 3,711,604, Jan. 16, 1973 to Colodney et al; U.S. Pat. No. 3,911,104, Oct. 7, 1975 to Harrison; U.S. Pat. No. 3,935,306, Jan. 27, 1976 to Roberts et al; and U.S. Pat. No. 4,040,858, Aug. 9, 1977 to Wason.

Application of fluoride ions to dental enamel serves to protect teeth against decay. A wide variety of fluoride ion-yielding materials can be employed as sources of soluble fluoride in the instant compositions. Examples of suitable fluoride ion-yielding materials are found in Briner et al; U.S. Pat. No. 3,535,421; issued Oct. 20, 1970 and Widder et al; U.S. Pat. No. 3,678,154; issued Jul. 18, 1972, both patents being incorporated herein by reference. Preferred fluoride ion sources for use herein include stannous fluoride, monofluorophosphate, sodium fluoride, potassium fluoride and ammonium fluoride. Sodium fluoride is particularly preferred. Preferably the instant compositions provide from about 50 ppm to 10,000 ppm, more preferably from about 100 to 3000 ppm, of fluoride ions in the aqueous solutions that contact dental surfaces when used with the strip of material used in the mouth.

Other anti-caries actives include xylitol.

4. Antimicrobial Actives

Antimicrobial actives can also be present in the oral care compositions. Such actives may include, but are not limited to, 5-chloro-2-(2,4-dichlorophenoxy)-phenol, commonly referred to as triclosan, and described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; phthalic acid and its salts including, but not limited to those disclosed in U.S. Pat. No. 4,994,262, Feb. 19, 1991, substituted monoperthalic acid and its salts and esters as disclosed in U.S. Pat. Nos. 4,990,329, Feb. 5, 1991, 5,110,583, May 5, 1992 and 4,716,035, Dec. 29, 1987, all to Sampathkumar; preferably magnesium monoperoxy phthalate, chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicyanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion actives; antibiotics such as augmentin, amoxicillin, tetracycline, doxycycline, minocycline, and metronidazole; and analogs and salts of the above; essential oils including thymol, geraniol, carvacrol, citral, hinokitiol, eucalyptol, catechol (particularly 4-allyl catechol), metals or metal ions (e.g., silver, copper, zinc, etc) and mixtures thereof; methyl salicylate; chlorite and metal salts of chlorite and mixtures of all of the above.

5. Anti-Inflammatory and Anti-Sensitivity Actives

Anti-inflammatory actives can also be present in the oral care compositions. Such actives may include, but are not limited to, non-steroidal anti-inflammatory actives or NSAIDs such as ketorolac, flurbiprofen, ibuprofen, naproxen, indomethacin, aspirin, ketoprofen, piroxicam and meclofenamic acid. Use of NSAIDs such as Ketorolac are claimed in U.S. Pat. No. 5,626,838, issued May 6, 1997, herein incorporated by reference. Disclosed therein are methods of preventing and, or treating primary and reoccurring squamous cell carcinoma of the oral cavity or oropharynx by topical administration to the oral cavity or oropharynx an effective amount of an NSAID.

Anti-sensitivity actives can include potassium nitrate, clove oil (Eugenol) and other herbal or flavor actives/agents.

6. Nutrients

Nutrients may improve the condition of the oral cavity and can be included in the oral care compositions. Nutrients include minerals, vitamins, oral nutritional supplements, enteral nutritional supplements, and mixtures thereof.

Minerals that can be included with the compositions of the present invention include calcium, phosphorus, fluoride, zinc, manganese, potassium and mixtures thereof. These minerals are disclosed in Drug Facts and Comparisons (loose leaf drug information service), Wolters Kluer Company, St. Louis, Mo., ©1997, pp 10-17; incorporated herein by reference.

Vitamins can be included with minerals or used separately. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Such vitamins are disclosed in Drug Facts and Comparisons (loose leaf drug information service), Wolters Kluer Company, St. Louis, Mo., ©1997, pp. 3-10; incorporated herein by reference.

Oral nutritional supplements include amino acids, lipotropics, fish oil, and mixtures thereof, as disclosed in Drug Facts and Comparisons (loose leaf drug information service), Wolters Kluer Company, St. Louis, Mo., ©1997, pp. 54-54e; incorporated herein by reference. Amino acids include, but, are not limited to L-Tryptophan, L-Lysine, Methionine, Threonine, Levocamitine or L-carnitine and mixtures thereof. Lipotropics include, but, are not limited to choline, inositol, betaine, linoleic acid, linolenic acid, and mixtures thereof. Fish oil contains large amounts of Omega-3 (N-3) Polyunsaturated fatty acids, eicosapentaenoic acid and docosahexaenoic acid.

Entenal nutritional supplements include, but, are not limited to protein products, glucose polymers, corn oil, safflower oil, medium chain triglycerides as disclosed in Drug Facts and Comparisons (loose leaf drug information service), Wolters Kluer Company, St. Louis, Mo., ©1997, pp. 55-57; incorporated herein by reference.

7. Enzymes

An individual or combination of several compatible enzymes can be included in the oral care composition. Enzymes are biological catalysts of chemical reactions in living systems. Enzymes combine with the substrates on which they act forming an intermediate enzyme-substrate complex. This complex is then converted to a reaction product and a liberated enzyme which continues its specific enzymatic function.

Enzymes provide several benefits when used for cleansing of the oral cavity. Proteases break down salivary proteins which are absorbed onto the tooth surface and form the pellicle; the first layer of resulting plaque. Proteases along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only present plaque formation, but also prevent the development of calculus by breaking-up the carbohydrate-protein complex that binds calcium, preventing mineralization.

Enzymes useful in the present invention include any of the commercially available proteases, glucanohydrolases, endoglycosidases, amylases, mutanases, lipases and mucinases or compatible mixtures thereof. Preferred are the proteases, dextranases, endoglycosidases and mutanases, most preferred being papain, endoglycosidase or a mixture of dextranase and mutanase. Additional enzymes suitable for use in the present invention are disclosed in U.S. Pat. No. 5,000,939 to Dring et al., Mar. 19, 1991; U.S. Pat. No. 4,992,420 to Neeser, Feb. 12, 1991; U.S. Pat. No. 4,355,022 to Rabussay, Oct. 19, 1982; U.S. Pat. No. 4,154,815 to Pader, May 15, 1979; U.S. Pat. No. 4,058,595 to Colodney, Nov. 15, 1977; U.S. Pat. No. 3,991,177 to Virda et al., Nov. 9, 1976 and U.S. Pat. No. 3,696,191 to Weeks, Oct. 3, 1972; all incorporated herein by reference.

8. Mouth and Throat Products

Other materials that can be used with the present invention include commonly known mouth and throat products. Such products are disclosed in Drug Facts and Comparisons (loose leaf drug information service), Wolters Kluer Company, St. Louis, Mo., ©1997, pp. 520b-527; incorporated herein by reference. These products include, but, are not limited to anti-fungal, antibiotic and analgesic actives.

9. Antioxidants

Antioxidants are generally recognized as useful in compositions such as those of the present invention. Antioxidants are disclosed in texts such as Cadenas and Packer, The Handbook of Antioxidants, © 1996 by Marcel Dekker, Inc., incorporated herein by reference. Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof.

10. H-2 Antagonists

Histamine-2 (H-2 or H2) receptor antagonist compounds (H-2 antagonists) may be used in the oral care composition of the present invention. As used herein, selective H-2 antagonists are compounds that block H-2 receptors, but do not have meaningful activity in blocking histamine-1 (H-1 or H2) receptors. Selective H-2 antagonists stimulates the contraction of smooth muscle from various organs, such as the gut and bronchi; this effect can be suppressed by low concentrations of mepyramine—a typical antihistaminic drug. The pharmacological receptors involved in these mepyramine-sensitive histamine responses have been defined as H-1 receptors (Ash, A. S. F. & H. O. Schild, Brit. J. Pharmacol Chemother., Vol. 27 (1966), p. 427, incorporated herein by reference). Histamine also stimulates the secretion of acid by the stomach (Loew, E. R. & O. Chickering, Proc. Soc. Exp. Biol. Med., Vol. 48 (1941), p. 65, incorporated herein by reference), increases the heart rate (Trendelenburg, U., J. Pharmacol., Vol. 130 (1960), p. 450, incorporated herein by reference), and inhibits contractions in the rat uterus (Dews, P. B. & J. D. P. Graham, Brit. J. Pharmacol. Chemother., Vol. 1 (1946), p. 278, incorporated herein by reference); these actions cannot be antagonized by mepyramine and related drugs. The H-2 antagonists useful in the oral care compositions or substances are those that blockade the receptors involved in mepyramine-insensitive, non-H-1 (H-2), histamine responses, and do not blockade the receptors involved in mepyramine-sensitive histamine responses.

Selective H-2 antagonists are those compounds found to be H-2 antagonists through their performance in classical preclinical screening tests for H-2 antagonist function. Selective H-2 antagonists are identified as compounds which can be demonstrated to function as competitive or non-competitive inhibitors of histamine-mediated effects in those screening models specifically dependent upon H-2 receptor function, but to lack significant histamine antagonist activity in those screening models dependent upon H-1 receptor function. Specifically, this includes compounds that would be classified as described by Black, J. W., W. A. M. Duncan, C. J. Durant, C. R. Ganellin & E. M. Parsons, “Definition and Antagonism of Histamine H@-Receptors”, Nature, Vol. 236 (Apr. 21, 1972), pp. 385-390 (Black), incorporated herein by reference, as H-2 antagonists if assessed as described by Black through testing with the guinea pig spontaneously beating right atria in vitro assay and the rat gastric acid secretion in vivo assay, but shown to lack in significant H-1 antagonist activity relative to H-2 antagonist activity, if assessed as described by Black with either the guinea pig ileum contraction in vitro assay or the rat stomach muscle contraction in vivo assay. Preferably selective H-2 antagonists demonstrate no significant H-1 activity at reasonable dosage levels in the above H-1 assays. Typical reasonable dosage level is the lowest dosage level at which 90% inhibition of histamine, preferably 99% inhibition of histamine, is achieved in the above H-2 assays.

Selective H-2 antagonists include compounds meeting the above criteria which are disclosed in U.S. Pat. Nos. 5,294,433 and 5,364,616 Singer et al., issued Mar. 15, 1994 and Nov. 15, 1994 respectively and assigned to Procter & Gamble; both herein incorporated by reference. wherein the selective H-2 antagonist is selected from the group consisting of cimetidine, etintidine, ranitidine, ICIA-5165, tiotidine, ORF-17578, lupitidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, and HB-408. 4. Particularly preferred is cimetidine (SKF-92334), N-cyano-N′-methyl-N″-(2-(((5-methyl-1H-imidazol-4-yl)methyl)thio)ethyl)guanidine:

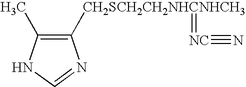

Cimetidine is also disclosed in the Merck Index, 11th edition (1989), p. 354 (entry no. 2279), and Physicians' Desk Reference, 46th edition (1992), p. 2228. Related preferred H-2 antagonists include burimamide and metiamide.

The oral care composition (and the barrier composition) can also include gelling agents and other ingredients that may be the same as or similar to those disclosed for the tooth whitening oral care composition. In one embodiment, the tooth whitening oral care composition and the oral care composition comprise different gelling agents or adhesion agents, or combinations thereof, in order to impart different adhesive characteristics to the tooth whitening oral care composition and the oral care composition. While the oral care actives have been described as forming part on oral care composition that is disposed apart from the tooth whitening oral care composition, it is contemplated that the oral care active can be combined with tooth whitening composition and/or the tooth whitening composition can deliver oral benefits in addition to tooth whitening or bleaching. For instance, the oral care active can be mixed with the material that forms part of the mesh or scrim in the tooth whitening composition, or the oral care active can be admixed with the strip of material.

While the above-described oral care compositions may be formed from the ingredients listed above, it will be appreciated that, after manufacture, the oral care composition may or may not contain these exact ingredients due to changes or reactions that may occur during the manufacture of the oral care composition.

Methods of Use

A safe and effective amount of the compositions of the present invention may be topically applied to the mucosal tissue of the oral cavity, to the gingival tissue of the oral cavity, and/or to the surface of the teeth, for the treatment or prevention of the above mentioned conditions in several ways.

The product 10 and/or 20 can be applied to the teeth and/or soft tissue for between about 1 minute and about 8 hours. In some embodiments, it may be desirable to follow a regimen where the product is applied for greater than about 1, 2, 3, 4, 5, 6, 7, 8, 9, 10, 15, 20, 30, 40, 50, 60, 90, 120, 150, 180, 210, 240, 270, 300, 330, 360, 390, 420, 450, 480 minutes and/or less than 480, 450, 420, 390, 360, 330, 300, 270, 240, 210, 180, 150, 120, 90, 60, 50, 40, 30, 20, 15, 10, 9, 8, 7, 6, 5, 4, 3, or 2 minutes and any combination thereof, wherein the oral care active has a concentration between about 1% and about 50% by weight of the oral care composition. Such a regimen could be advantageously used once a day for greater than about one month, two months, four months, six months, twelve months, eighteen months, two years, five years, eight years, ten years and/or less than about fifteen years, ten years, eight years, five years, two years, 18 months, 12 months, six months, four months, two months, one month and any combination thereof. In another embodiment such a regimen could be advantageously used once a day for greater than about one month and less than about 5 years. A kit could be provided that contains a one month, two month, three month, four month, five month, six month, or 12 month supply of the product 10 and/or 20 for both the upper and lower dentition. The product 10 and/or 20 can be packaged, such as in a pouch, and, in turn, a plurality of the packaged products 10 and/or 20 can be provided in a storage container or outer package or carton, such as shown by way of example in U.S. Pat. No. D496,495. Two strips of material comprising one or more oral care compositions, one for an upper dentition, and one for lower dentition can be contained within a single package, such as a pouch. Such a regimen could also be employed for use in the shower either in the morning or evening, wherein the product is worn during showering or bathing.

In another embodiment, the product may be applied for greater than about 1, 2, 3, 4, 5, 10, 15, 20, 25, 30, or 35 and/or less than about 60, 55, 50, 45, or 40 minutes any combination thereof, wherein in some embodiments the concentration of the oral care active is greater than 5% by weight of the oral care composition and/or the oral care active is less than about 10% by weight of the oral care composition. The product 10 and/or 20 can be packaged in a pouch or other container. A sufficient number of packaged products can be provided as a kit. The kit may contain instructions describing the regimen for using the packaged products. The number of packaged products in a kit can vary widely, but may be between about 6 and about 200 depending upon the regimen. In one embodiment the kit comprises greater than about 10, 20, 30, 40 and less than about 200, 100, 50, 45, 40 packaged products and/or any combination thereof, each of the packaged products comprising two strips of material comprising oral care compositions, one sized to fit the upper dentition and one sized to fit the lower dentition. Such a regimen could be advantageously used once a day for greater than about one month, one and a half months, two months, four months, six months, twelve months, eighteen months, two years, five years, eight years, ten years and/or less than about fifteen years, ten years, eight years, five years, two years, 18 months, 12 months, six months, four months, two months, one and a half months and any combination thereof. Typically, product 10 and/or 20 will be provided for both the upper and lower maxillary within a kit. Each application of a product to the teeth may utilize a new or fresh product from the kit.

In yet another embodiment of the present invention, a kit contains a plurality of products 10 and/or 20, wherein each has a different concentration of the oral care active. The concentration of the active may range within each kit between about 1% and about 25% by weight of the oral care composition. The concentration difference between the packaged products within a kit may be greater than about 1, 2, 3, 4, 5, 6, 10, 15, 20% and or less than about 20, 15, 10, 9, 8, 7, 6, 5, or 4% by weight of the oral care composition and any combination thereof. For instance, the kit might contain one or more of the following: a first product having a concentration of about 4% hydrogen peroxide by weight of the oral care composition, a second product 10 and/or 20 having a concentration of about 8% hydrogen peroxide by weight of the oral care composition, a third product having a concentration of about 12% hydrogen peroxide by weight of the oral care composition, and a fourth product having a concentration of about 16% hydrogen peroxide by weight of the oral care composition. The products are used in order of increasing concentration of the oral care active. For example, a user would use, in order, the first, second, third, and fourth products. This method can decrease tooth sensitivity associated with application of products containing higher concentrations of an oral care active, such as hydrogen peroxide. The packages containing the first, second, third, and fourth products within the kit can be textually or graphically coded to indicate their preferred order of use (e.g., color coded or numerically labeled).

It may desirable to apply a product having the combination of a tooth whitening active and an oral care active at least about 1, 2, 3, 4, 5, 6, 7, or 8 and/or less than about 20, 18, 16, 14, 12, or 10 times for a regimen associated with a single kit that may last greater than about 1 day and/or less than about 5 years, wherein a new product may be applied to the teeth about 1, 2, 3, 4, and/or 5 times per day and any combination thereof.

It may also be desirable to apply an oral care product comprising an oral care active, wherein the active is a peroxide composition. These products comprising peroxide can be applied to the teeth and/or the soft tissues of the oral cavity. These products can be applied from about 1, 2, 3 and or less than about 3, 2 times per day to the desired surface of the oral cavity. In one embodiment the product is applied the lingual and facial surfaces of a plurality of teeth. In another embodiment the product is applied to the gum area only, in yet another embodiment the product is applied to the teeth and the gum area. This product can be applied to the oral surface for greater than about 1, 2, 3, 4, 5 6, 7, 8, 9, 10 minutes and less than about 10, 9, 8, 7, 6, 5, 4, 3, 2, minutes and any combination thereof. Such a regimen could be advantageously at least once a day for greater than about one month, two months, four months, six months, twelve months, eighteen months, two years, five years, eight years, ten years and/or less than about fifteen years, ten years, eight years, five years, two years, 18 months, 12 months, six months, four months, two months, one month and any combination thereof. A kit can comprise a one month, two month, three month, four month, five month, six month, or 12 month supply of the product 10 and/or 20 for both the upper and lower dentition. In one embodiment a kit comprises greater than about 10, 20, 30, 40, 50, 60, 70, 80, 90, 120 strips and/or less than about 730, 365, 200, 185, 150, 120, 90, 80, 70, 60, 50, 40, 30, 20 strips and any combination thereof.

All documents cited in the Detailed Description of the Invention are, in relevant part, incorporated herein by reference; the citation of any document is not to be construed as an admission that it is prior art with respect to the present invention.

It is significant to note that any of the features, aspects, or details of any method and/or product described herein can be combined, either entirely or partially, with any other feature, aspect, or detail of one or more other methods or products described herein. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

What is claimed is:

1. A method for delivering one or more oral care benefits to the surfaces of the oral cavity comprising:
applying a product comprising a substrate and an oral care composition comprising an oral care active to a plurality of teeth and adjacent soft tissue at least once every day for at least about five minutes per application, wherein the product is applied to the teeth daily and adjacent soft tissue for greater than about six months and wherein a fresh product is applied with each use.

2. The method of claim 1, wherein said oral care active is a tooth whitening active.

3. The method of claim 2, wherein said tooth whitening active is peroxide.

4. The method of claim 3, wherein said oral care composition comprises less than 7.5% peroxide by weight of the total composition, and has a peroxide density of less than about 1.3 mg/cm².

5. The method of claim 4, wherein said oral care composition comprises greater than about 6.5% peroxide by weight of the total composition and less than 7.5% peroxide by weight of the total composition, and has a peroxide density of less than about 1.3 mg/cm².

6. The method of claim 4, wherein said oral care composition comprises greater than about 6% peroxide by weight of the total composition and less than about 6.5% peroxide by weight of the total composition, and has a peroxide density of less than about 1.3 mg/cm².

7. The method of claim 4, wherein said oral care composition comprises less than 6.0% peroxide by weight of the total composition, and has a peroxide density of less than about 1.3 mg/cm².

8. The method of claim 1, wherein said oral care product is applied to the teeth and adjacent soft tissue at least once daily for greater than about one year.

9. A method of reducing tartar on the hard surfaces of the oral cavity comprising:
applying a product comprising a substrate and one or more oral care compositions to the teeth and adjacent soft tissue, wherein at least one of said oral care compositions comprises an anti-tartar active at least once a day for at least about five minutes per application, wherein the product is applied to the teeth and adjacent soft tissue daily for greater than about one month, and wherein a fresh product is applied with each use.

10. The method of claim 9, wherein said product is applied to the teeth and adjacent soft tissue daily for greater than about two months.

11. The method of claim 9, wherein said product is applied to the teeth and adjacent soft tissue daily for greater than about three months.

12. The method of claim 9, wherein said product is applied to the teeth and adjacent soft tissue daily for greater than about six months.

13. The method of claim 9, wherein at least one of said oral care compositions comprises a tooth whitening active.

14. The method of claim 13, wherein said tooth whitening active is peroxide.

15. The method of claim 13, wherein at least one of said oral care compositions comprises both said tooth whitening active and said anti-tartar active.

16. The method of claim 15, wherein said oral care composition comprises from about 1% to about 30% of said tooth whitening active by weight of the total composition and from about 0.5% to about 3% of said anti-tartar active by weight of the total composition.

17. The method of claim 16, wherein said oral care composition comprises from about 1% to about 10% of said tooth whitening active by weight of the total composition and from about 1% to about 3% of said anti-tartar active by weight of the total composition.

18. The method of claim 17, wherein said tooth whitening active is peroxide, and wherein said anti-tartar active is a phosphate.

19. The method of claim 18, wherein said anti-tartar active is a pyrophosphate.

20. The method of claim 13 wherein said product comprises two oral care compositions, wherein one of said oral care compositions comprises said tooth whitening active, and one of said oral care compositions comprises said anti-tartar active, wherein said oral care compositions are separated by a barrier composition.
